(19) United States
(12) Patent Application Publication
(10) Pub. No.: US 2008/0132899 A1
(43) Pub. Date: Jun. 5, 2008

(54) Composite implant and method for treating bone abnormalities

(76) Inventors: John H. Shadduck, Tiburon, CA (US); Csaba Truckai, Saratoga, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/130,837

(22) Filed: May 17, 2005

Related U.S. Application Data
(60) Provisional application No. 60/571,753, filed on May 17, 2004.

(51) Int. Cl. A61B 17/58

(52) U.S. Cl. 606/94; 606/92; 606/93

(57) ABSTRACT

This invention relates to implantable bone fill materials, systems and methods of treating bone abnormalities such as compression fractures of vertebrae, bone necrosis, bone tumors, cysts and the like. In an exemplary embodiment, the bone abnormality is accessed and a space is created by bone removal or compaction. An exemplary implant of the invention has a substantially fluid impermeable surface portion and an interior portion including an in-situ hardenable bone cement. The method of the invention includes applying energy to the fill material to accelerate polymerization and hardening of the material for supporting the bone.

### CROSS-REFERENCE TO RELATED APPLICATIONS

This application claims benefit of Provisional U.S. Patent Application Ser. No. 60/571,753 filed May 17, 2004 (Docket No. S-7700-010) titled Composite Implant and Method for Treating Bone Abnormalities, which is incorporated herein by this reference.

### BACKGROUND OF THE INVENTION

### Field of the Invention

This invention relates to implants and methods for treating abnormalities in bones such as a compression fracture of a vertebra. More in particular, the invention relates to a plurality of elongated, flexible sleeve-like members that can be packed and entangled in a bone and wherein the members have fluid impermeable surfaces with an interior core that includes materials that can be polymerized in situ to provide a rigid bone support structure.

### SUMMARY OF THE INVENTION

The invention provides a method of correcting bone abnormalities including bone tumors and cysts, avascular necrosis of the femoral head, tibial plateau fractures and compression fractures of the spine. In an exemplary embodiment, the system of the invention provides flexible filament-like structures that are packed into a bone that are controllably hardened by application of energy from a remote source to cause in-situ polymerization and hardening of materials within the filament-like structures.

The abnormality may be corrected by first accessing and boring into the damaged tissue or bone and reaming out the damaged and/or diseased area using any of the presently accepted procedures, or the damaged area may be prepared by expanding a structure within the damaged bone to compact cancellous bone. After removal and/or compaction of the damaged tissue, the bone can be stabilized with the in-situ polymerized filament-like structures.

### BRIEF DESCRIPTION OF THE DRAWINGS

In the following detailed description, similar reference numerals are used to depict like elements in the various figures.

FIG. 1 is a side view of a segment of a spine with a vertebra having a compression fracture that can be repaired with the present invention, showing an introducer in a method of the invention for creating at least one space in the vertebral body.

FIG. 2 is a cross-sectional view of the vertebra and abnormality of FIG. 1 with spaces created therein by cutting, reaming or compaction means.

FIGS. 3A-3C are cross-sectional views of exemplary polymerizable element corresponding to the invention;

FIG. 3A illustrating an ultrasound fragmentable barrier between first and second components of a bone cement such as PMMA;

FIG. 3B illustrating a cross-sectional view of a sleeve with fragmentable microspheres containing first and second components of a bone cement; and

FIG. 3C illustrating a member with a bore extending therethrough for receiving an elongated ultrasound device for mixing polymerizable components.

FIG. 4 is a perspective view of a portion of an alternative implant for creating an in-situ hardenable bone support material.

FIG. 5 is a cross-sectional view of a portion of the bone support material of FIG. 4.

FIG. 6 is a cut-away view of a portion of an alternative structure for containing an in-situ hardenable bone cement.

FIG. 7 is a schematic view of the use of an implant as in FIG. 6 or preventing extravasion of an in-situ hardenable bone cement.

### DETAILED DESCRIPTION OF THE INVENTION

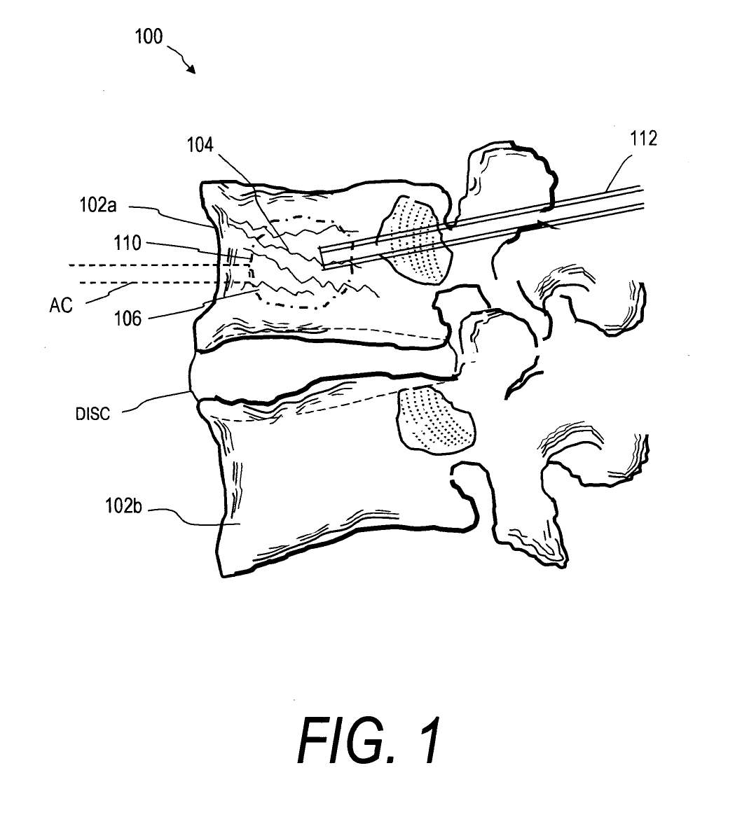

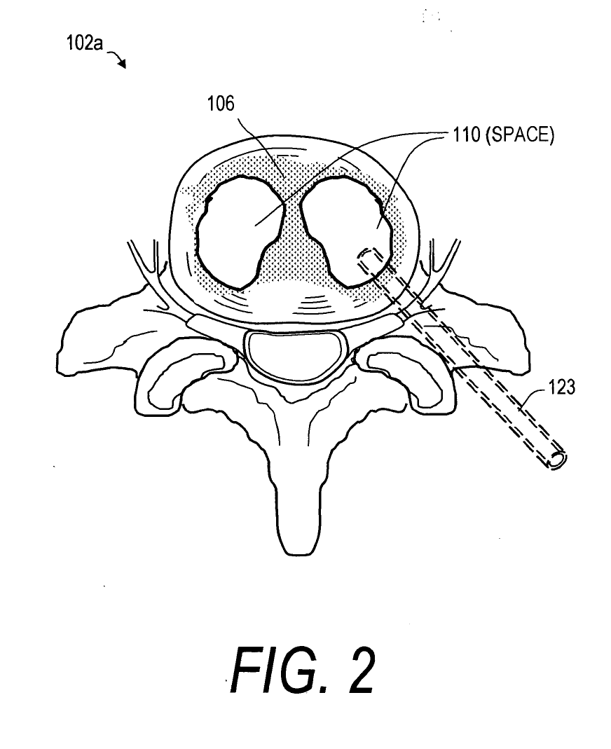

FIG. 1 shows a spine segment 100 wherein vertebra 102a has a compression fracture 104 that has caused a collapse in vertebral height. The cancellous bone 106 in the interior of the vertebra has been treated to provide a cavity or space 110 therein. The space or spaces 110 as indicated in FIG. 2 can be created with cutting and extraction tools indicated at 112 in FIG. 1, compaction of cancellous bone by means of ultrasound or a low frequency mechanical vibrating device, compaction by a balloon, or the space can created by a combination of compaction and cutting. There can be a single space or a multiplicity of spaces 110 as in FIG. 2.

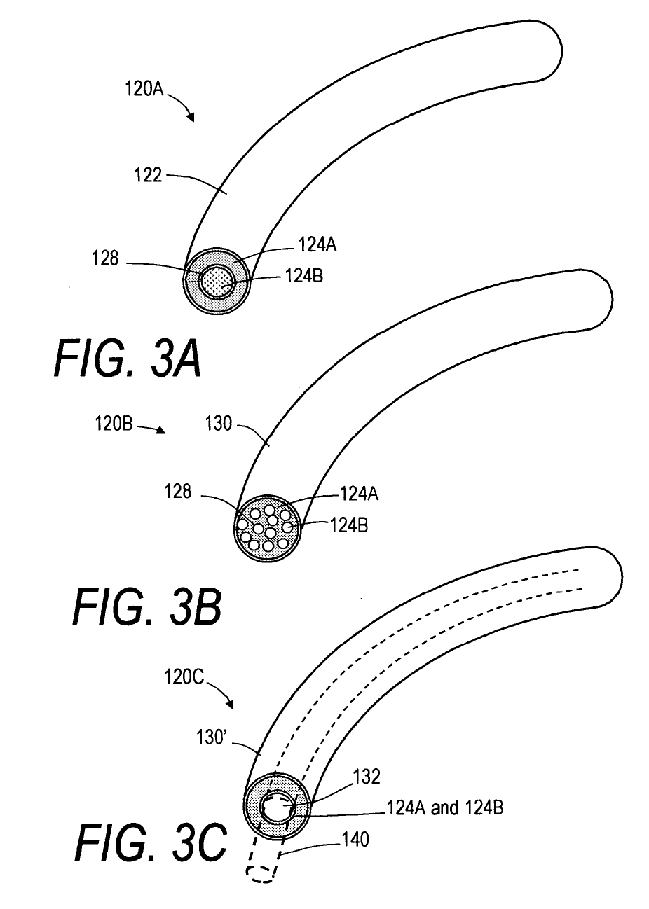

FIG. 3A illustrates an exemplary implant body 120A of the invention that comprises a polymer sleeve or monolithic polymer body that has a substantially fluid impermeable surface 122. The body has an initial flexible, deformable condition, similar to a monofilament fish line, that is stiff enough to allow it to be pushed and thereafter packed and convoluted into cavity 110 in a bone after being introduced through the bore of a small diameter sleeve 123 (see FIG. 2). The interior of body 120A carries first and second polymerizable components 124A and 124B that when exposed to one another cause a chemical interaction to thereby polymerize the components and alter the implant body to a inflexible, high modulus, substantially rigid and non-deformable condition.

In one embodiment, the implant body 120A has a surface 122 of any biocompatible polymer 125 (e.g., Teflon, Dacron, silicone rubber, polysulfone, etc.) and the polymerizable components 124A and 124B are independent agents that separated in by an intermediate sacrificial element 128 that when perforated, obliterated or sacrificed allows the components to intermix to cause a chemical reaction process herein described generally as a polymerization process (see FIG. 3A). For example, the polymerizable components can be PMMA precursors. This polymerization process allows the body 120A to transform irreversibly into its rigid or inflexible state. In body 120A of FIG. 3A, the polymerizable components 124A and 124B are in concentric sleeve portions separated by sacrificial or disintegratable element 128. The element 128 preferably disintegrates in response ultrasound energy, although other stimulus such as heat caused by an Rf or light source are also possible. Rf can be coupled to element 128 by electrical leads wherein the element has conductive particles of microfilaments therein to allow heating and melting of the element 128. In FIG. 3B, either or both polymerizable components 124A and 124B are microencapsulated wherein the disintegrable element 128 comprises the polymer of the capsule material. The sacrificial element 128 preferably is sacrificial in response ultrasound energy or heat from inductive heating as in known in the art. The fragmentable component 128 can carry any suitable biocompatible material that cooperates with photonic energy, electrical energy or magnetic energy to elevate its temperature. Light sources, Rf sources and magnetic emitters are known and can be used to deliver energy to the implant, e.g., as disclosed in the author's U.S. patent application Ser. No. 09/473,371 filed Dec. 27, 1999 (now U.S. Pat. No. 6,306,075), incorporated herein by reference. The detail of the energy source need not be further described herein. In FIGS. 3A-3B, the member is illustrated as having an exterior sleeve, but it should be appreciated that the member can be a solid low-modulus polymer with the first and second polymerizable materials dispersed therein together with a surface modification that makes the surface substantially fluid-impermeable.

Still other alternative embodiments are contemplated wherein the energy can be light energy, microwave energy, inductive heating energy, mechanical forces, shear forces, tension forces and/or changes in pressure.

In the embodiments of FIGS. 3A-3B, the polymerization process results in a hardened material such as PMMA in the interior of the implant body. PMMA can be provided as a two-part mixable polymer with one powder component and one liquid component. The scope of the invention includes any polymer precursors 124A and 124B that yield a rigid biocompatible polymer. When such components are carried in an implant body 120A, the compositions that are polymerizable to make the body rigid preferably comprise at least about 50% of the body by volume, and more preferably at least about 80% by volume. The body 120A can be provided in selected lengths—or can be cut to length after introducing into a bone cavity 110 (see FIG. 2). The lengths of each implant body can be from about 5 mm to 100 mm. or more.

FIG. 3B illustrates an implant body 120B comprises a thin, closed-end sleeve 130 that carries any polymer precursors 124A and 124B that when exposed to one another result in a polymerization process that yields a rigid biocompatible polymer. Again, PMMA can be provided as a two-part mixable polymer with one powder component and a microencapsulated liquid component, with both components carried in the sealed, closed-end sleeve 130. The sleeve 130 has a substantially fluid impermeable surface to thereby provide means to assure that the polymer cannot leak out during the polymerization process. Again, the implant body 120B has an initial flexible state for introduction and packing into space 110 in a bone. In this embodiment, the microencapsulated components (either 124A or 124B) can have any form of sacrificial surface 128 that when perforated, obliterated or sacrificed allows the components to intermix to cause the polymerization process to irreversibly rigidify the body. It should be appreciated that microencapsulation of polymerizing components is one preferred aspect of invention, any other sacrificial elements intermediate the first and second components 124A and 124B within body 120 are possible, such as an element that extends at least one of axially, cross-axially, or helically or concentrically as described above at any microscale or macroscale.

FIG. 3C illustrates an alternative preferred implant body 120C that comprises a thin-wall, closed-end sleeve 130′ that has a passageway 132 extending therethrough for receiving an energy delivery member 140 (phantom view) such as a ultrasound member. In all other respects, the implant body is as described previously. In preferred embodiments, the sleeve 130 or 130′ is of a substantially non-distensible polymer.

In a preferred embodiment, the implant body (120A-120C) has a mean cross section of less that 5 mm. More preferably, the implant body a mean cross section of less that about 4 mm. but can have any suitable cross section.

In preferred embodiments, the polymer of the implant body or sleeve (130 or 130′) and/or at least one of the first and second components 124A and 124B carries filaments for reinforcing the rigid state of the implant following the polymerization process. The filaments or fibers can be any suitable reinforcing material known in the art such as Kevlar, carbon fiber, titanium stainless steel or another metal.

In another embodiment, the implant body 120C can include a sleeve wall 130′ (FIG. 3C) comprising knit, woven or braided filaments 144 as is known in the art of fabricating biocompatible implants.

In preferred embodiments, the body 120A-120C can carry additional filler materials that are responsive to energy delivery to cause agitation, mixing, stirring, turbulent flows and the like to cause optimal chemical interaction of the components that form a hardened material. In one embodiment, a filler comprising an ultrasound responsive material such as high-impedance compositions or elements that wiggle (e.g., a metal or ceramic element) or an encapsulated gas that cavitates, explodes or the like. The filler can also comprise elements that are magnetically responsive to an oscillating magnetic source to allow agitation and mixing of polymerizing components. Electrical energy from an Rf source can be used to agitate and heat the polymerizing components by means of conductive fillers in the hardenable material. The conductive filler comprises filaments, particles, microspheres, powders or crystals. The conductive filler is a least one of titanium, tantalum, stainless steel, silver, gold, platinum, nickel, tin, nickel titanium alloy, palladium, magnesium, iron, molybdenum, tungsten, zirconium, zinc, cobalt, chromium or carbon.

In preferred embodiments, the implant body 120A-120C includes at least portions of the surface material polymer that are bioerodible, biodegradable, bioabsorbable and/or bioexcretable. By this means, natural bone infill can migrate into the body after implantation.

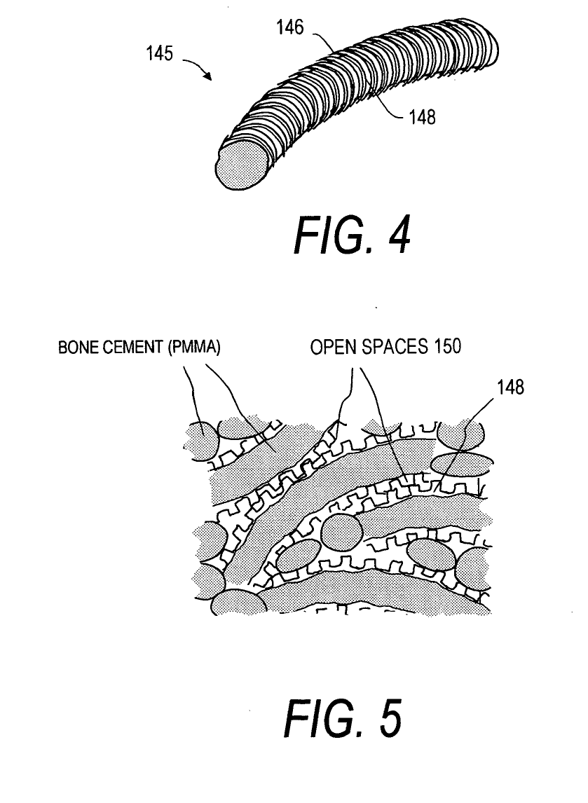

In another embodiment shown in FIG. 4, each polymer implant body 145 has a non-smooth exterior surface 146 with an interior volume of hardenable bone cement so that when the bodies are compacted together into cavity 110 in a bone (FIG. 5) there will exist open spaces 150 between the surfaces 146 that will remain open for natural bone ingrowth. For example, each implant body can have a grooved exterior surface 148 with the polymer surface having a suitable modulus that will prevent collapse of the grooved structure. The grooved exterior surface can have grooves that extend at least one of axially, helically and circumferentially. In general, the polymer body 145 has substantial surface relief elements for providing interstitial spaces after packing of at least one polymer structure in a cavity 110 in a bone.

In any embodiment, the implant body 120 further carries a radiopaque composition in either the polymer body or in at least one of the first and second components 124A and 124B.

FIG. 2 illustrates a procedure wherein at least one implant body (120A-120C) is injected and packed into space 110. Of particular interest, energy is applies to the implant body to irreversibly transform the volume into a polymerized composite that has high strength to support bone. Embodiments with reinforcing fibers can reduce a fracture, such as a vertebral fracture, in a manner that is improved over the injection of a conventional bone cement. The system of the invention entirely eliminates the possibility of cement and monomer extravasion (leakage) from the targeted interior region of the bone. Of particular interest, the implant prevents leakage of bone cement precursors that can damage nerves or migrate into the patient's blood stream.

It should be appreciated that the implant body can carry a polymer together with any of the following: cortical bone material, synthetic cortical replacement material, demineralized bone material, autograft and allograft materials. The implant body also can include drugs and agents for inducing bone growth, such as bone morphogenic protein.

The inventive implant bodies 120A-120C of FIGS. 3A-3C can have any suitable length. Substantially short implants may be injected toward the end of the procedure to assure the correct selection of total volume. Alternatively, the bodies can be a continuous element that is injected with the trailing end cut when the space 110 is filled. Such a continuous body with microencapsulating polymerizing elements 124A and 124B with a substantially solid polymer body can be compared to self-healing plastics that carry such microencapsulated agents for polymerizing following shear stresses.

In another embodiment, at least the exterior surface layer of the implant carries polymers or polymer precursors that cross-link and fuse together after the body is packed and convoluted in a bone cavity 110 (FIG. 5). For example, such surface cross-linking can be caused by a selected temperature increase that results from the polymerization process of PMMA or another bone cement that is ongoing in the interior of the implant.

As can be seen in FIG. 5, the packed, convoluted implants when in a final state in a bone cavity comprises a composite monolith that includes networked open spaces 150 and also includes a fiber-reinforced, high strength bone cement contained with a fluid impermeable polymer sleeve 125, 130, 130′. Of particular interest, the PMMA or other similar high strength polymer at all times is contained within the fluid impermeable surfaces to prevent contact with nerves and the patient's blood stream.

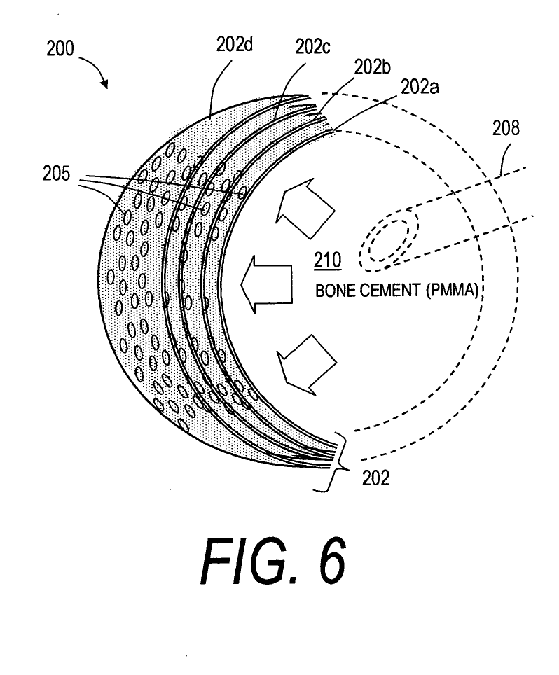

FIG. 6 illustrates an alternative implant 200 and aspect of the invention for controlling extravasion of cement and monomers of bone cement when treating bone. As can be seen in the cut-away view of FIG. 6, the implant body 200 comprises a plurality of distensible or non-distensible concentric, fluid permeable shell elements 202a-202d (collectively 202) that can be collapsed, crushed or rolled into a compact form for introduction into bone through a small diameter introducer. The shell elements can number from about 2 to 20 and are substantially porous with pores 205 therein. The shell elements can be perforated, knit, woven, braided and an combination thereof. Suitable biocompatible materials are nylons, urethanes, Dacron and Teflons. In one aspect of the invention, the plurality of shell elements are inserted into a bone space 110 (cf. FIG. 2), and then an, introducer 208 is penetrated through the shells 202 into an interior of the shell assembly. A radiopaque marker is carried on at least one interior shell. Thereafter, a bone cement such as PMMA is directly injected into the interior region 210 and the elements deform as expand to the irregular surface of bone space 110 (cf. FIG. 2). At the same, the shell elements overlap and cause limited registration of pores 205. As the shell elements 202 expand further, the outward pressures of the cement progressively cause the collective surfaces of the shell elements 202 to form a substantially fluid impermeable surface. The shell elements also can be inserted directly into cancellous bone an filled with a bon cement to provide a similar method fro preventing extravasion or leakage of bone cement.

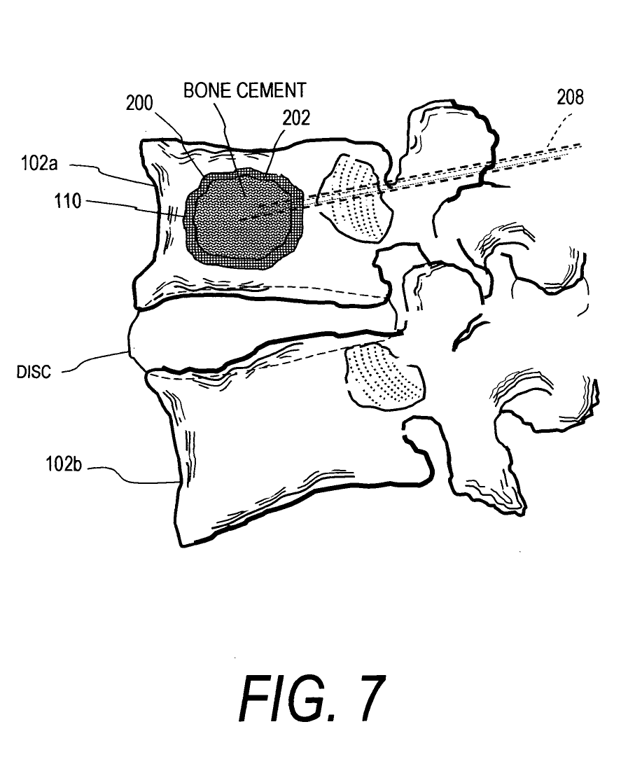

FIG. 7 illustrates the use of implant 200 of FIG. 6. The deformable implant is useful in containing bone cement within an assembly that becomes substantially fluid impermeable after filling with cement which prevents migration of the bone cement or monomers into the blood stream or into contact with nerves. The use of such elements solves a problem associated with prior art procedures wherein PMMA bone cement is injected directly into a bone cavity and its components can cause damage to nerves or can enter circulatory system and have serious consequences on the patient's health. While described in conjunction with a compression fracture of vertebra, it should be appreciated that the implant for sealing the extremities of a bone cavity from bone cement migration extends to similar uses in all bones such as the tibia and femur that are often treated with bone cement.

The implants of the invention also can carry suitable radiovisible elements, for example as longitudinal stripes, at ends of each elongate element or any other form. The implants also can carry any suitable pharmacological agent for immediate or timed release.

In another embodiment, a composite implant is provide that carries self-healing polymer components, for example microencapsulated components, that initiate a polymerization process when disrupted by shear forces in months or years following their implantation. The use of self-healing polymers has been proposed for polymer materials in industrial uses. The self-healing polymer implants of the invention are for the first time disclosed for use in a biomedical implant. Of particular interest, the self-healing polymer may be adapted for use in minimally invasive prophylactic procedures for needle injection into cancellous bone in elderly patients. Upon a compression fracture, the shear forces would release the self healing polymer to stabilize or support the bone defect.

The above description of the invention intended to be illustrative and not exhaustive. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

What is claimed:

1. A bone treatment method comprising the steps of:
(a) introducing into a bone a hardenable bone cement that includes an electrically conductive filler in a sufficient amount to allow ohmic heating of the bone cement; and
(b) delivering Rf energy to the bone cement wherein ohmic heating of the filler accelerates curing of the bone cement.

2. The bone treatment method of claim 1 wherein delivering Rf energy alters at least one the hardness, viscosity or elastic modulus of the bone cement.

3. The bone treatment method of claim 1 wherein step (a) introduces the bone cement within the interior of a deformable structure.

4. The bone treatment method of claim 3 wherein step (b) causes the deformable structure to become non-deformable.

5. A bone treatment method comprising the steps of:
(a) introducing a plurality of deformable elements into bone wherein each element includes a composition responsive to energy delivery from a remote energy source; and
(b) delivering energy to said composition wherein the response causes the elements to become non-deformable.

6. The method of claim 5 wherein delivering energy accelerates the polymerization of a bone cement.

7. The method of claim 5 wherein delivering energy sacrifices a barrier between first and second compositions to cause polymerization of said compositions.

8. The method of claim 5 wherein delivering energy sacrifices the surface of microcapsules carrying a polymerizing composition.

9. The method of claim 5 wherein delivering energy causes the elements to become non-deformable for reducing a supporting a fracture.

10. The method of claim 5 wherein delivering energy causes the elements to become non-deformable for supporting cortical bone.

11. The method of claim 5 wherein delivering energy includes delivering energy from at least one of a radiofrequency source, a light source, a microwave source and a magnetic energy source.

12. An implant system for treating a bone abnormality comprising at least one implant having a first flexible state, the structure having a substantially impermeable surface portion and an interior portion that responds to energy delivery for altering the implant to a second inflexible state, and energy source for delivering energy to the at least one implant.

13. The implant system of claim 12 wherein said interior portion includes an in-situ hardenable material.

14. The implant system of claim 13 wherein the hardenable material is at least one of PMMA, monocalcium phosphate, tricalcium phosphate, calcium carbonate, calcium sulphate or hydroxyapatite.

15. The implant system of claim 12 wherein said interior portion includes a sacrificial element that can be sacrificed in response to energy delivery.

16. The implant system of claim 11 wherein the sacrificial element separates first and second components of an in-situ hardenable material.

17. The implant system of claim 12 wherein hardenable material comprises at least one microencapsulated composition.

18. The implant system of claim 17 wherein each microcapsule includes a sacrificial surface that can be sacrificed in response to energy delivery.

19. The implant system of claim 12 wherein the energy source is at least one of a radiofrequency source, a laser source, a microwave source, a magnetic energy source.

20. A method of treating a bone a bone abnormality comprising:
(a) providing an implant body comprising a plurality concentric shells of a flexible, porous material;
(b) introducing the implant body into a bone in a collapsed condition; and
(c) injecting an in-situ hardenable bone fill material into an interior of the implant body thereby expanding the implant body wherein the shells substantially prevent extravasion of the fill material.

21. The method of claim 20 wherein step (a) provides shells of a perforated material.

22. The method of claim 20 wherein step (a) provides shells that are at least one of knit, woven or braided.

23. The method of claim 20 wherein step (a) provides shells that are of a polymer.
